(19) United States
(12) Patent Application Publication
(10) Pub. No.: US 2015/0111224 A1
(43) Pub. Date: Apr. 23, 2015

(54) BIOMARKERS FOR ADVERSE CARDIAC REMODELING

(71) Applicant: Fatih Arslan, Utrecht (NL)

(72) Inventor: Fatih Arslan, Utrecht (NL)

(21) Appl. No.: 14/385,445

(22) PCT Filed: Mar. 16, 2012
(86) PCT No.: PCT/NL2012/050166

§ 371 Date: Dec. 1, 2014

(51) Int. Cl. G01N 33/68

(52) U.S. Cl. CPC G01N 33/6893; G01N 2333/70596; G01N 2800/32; G01N 2800/52
USPC 435/7.21

(57) ABSTRACT

The present invention provides for certain biomarkers for adverse cardiac remodeling. The biomarkers have predictive value in assessing the risk of a subject developing heart failure and other conditions related to adverse cardiac remodeling, as well as diagnosing adverse cardiac remodeling, and determining response to therapy addressing adverse cardiac remodeling.

### FIELD OF THE INVENTION

The present invention is in the field of medicine, in particular in the field of cardiology. The invention provides certain biomarkers for adverse cardiac remodeling. The biomarkers have predictive value in assessing the risk of a subject developing heart failure and other conditions related to adverse cardiac remodeling, as well as diagnosing adverse cardiac remodeling, and determining response to therapy addressing adverse cardiac remodeling.

### INTRODUCTION

Ischemic heart disease is the largest socio-economic burden to Western societies. It becomes even a bigger problem in this era of rapid modernization of developing countries like China and India. The most severe and acute complication of ischemic heart disease is a heart attack, also known as myocardial infarction. In the USA, EU and Japan only, 2.4 million patients suffer from a myocardial infarction each year. The amount of money spent in the USA and the EU only for the treatment of ischemic heart disease exceeds [symbol] 150 billion every year. Unfortunately, infarct-related complications are increasing because more patients survive the initial life-threatening infarction, but have progressively worse cardiac function hereafter. Complications after myocardial infarction (MI) such as heart failure, fibrosis and arrhythmia result in high mortality rates and morbidity. The most important determinant of these complications is an improper cardiac repair response, referred to as adverse remodeling, adverse cardiac remodeling or adverse ventricular remodeling.

Heart failure (HF) has gained much attention, as it is the most severe and most frequent consequence of adverse remodeling after myocardial infarction. It is not without reason that the largest cardiology society in the world, the European Society of Cardiology (ESC), stated “HF is the epidemic of the 21^{st} century in Western societies”. In the USA, EU and Japan alone, at least 1.8 million patients are hospitalized with newly diagnosed infarct-related HF each year. The mortality rate is 20% within a year from diagnosis, while 50% die within 5 years. Quality of life of those that survive is severely affected as they suffer from progressively decreasing exercise tolerance and reduced capacity to conduct normal daily activities. The socio-economic burden is nearly [symbol] 60 billion annually for the USA and EU only, as a consequence of 1) the reduced exercise tolerance and subsequent reduced productivity, 2) expensive medical treatment that are not preventive but decrease symptoms and 3) hospital stays.

The healing of the infarcted heart is a very complex process involving many types of cells. Myocardial infarction is an acute event in which part of the heart muscle dies resulting in loss of pump function. Immediately after this acute event, repair processes are induced in the blood and the heart muscle characterized by enhanced inflammation. However, the type of inflammation determines whether the infarcted heart is repaired and remodeled properly. The key factor that drives improper healing and deleterious inflammation is the activation of innate immunity by molecules related to cardiac death and matrix degradation. In many patients, the immune system becomes activated in a detrimental way, resulting in inappropriate healing of the heart after myocardial infarction. In those cases, the heart will enter a process called adverse remodeling of the affected ventricle. Adverse remodeling has several deleterious consequences: heart failure, dilatation and fibrosis of the heart, disturbed contractility and relaxation, and disturbed electrical activation are known complications.

Current therapy for adverse cardiac remodeling-related complications such as heart failure is initiated after symptom onset, and per definition too late to reverse the adverse cardiac remodeling. As such, early detection of a risk of developing adverse cardiac remodeling would improve survival rate for subjects at risk. Therapy should be started before adverse cardiac remodeling results in disturbed heart function and geometry, heart failure, fibrosis, dilatation or ventricular arrhythmias.

Additionally, it would be useful if tools were available to monitor progression of adverse cardiac remodeling in such subjects, as well as response to therapy in such subjects.

### SUMMARY OF THE INVENTION

In a first aspect, the present invention relates to use of at least one biomarker selected from P-selectin glycoprotein ligand-1 (PSGL-1) and integrin-alpha4 (CD49d) in identifying a subject at risk of developing adverse cardiac remodeling, diagnosing a subject suffering from adverse cardiac remodeling, monitoring progression of adverse cardiac remodeling, and/or determining the response to therapy addressing adverse cardiac remodeling in a sample obtained from a subject.

Said adverse cardiac remodeling may result from myocardial infarction.

Said adverse cardiac remodeling may be indicative of the onset and/or occurrence of heart failure and/or remote myocardial fibrosis.

In a second aspect, the invention provides a method for identifying a subject at risk of developing adverse cardiac remodeling and/or diagnosing a subject suffering from adverse cardiac remodeling, said method comprising the steps of:

a) providing a sample of said subject;

b) determining the level of at least one biomarker selected from P-selectin glycoprotein ligand-1 (PSGL-1) and integrin-alpha4 (CD49d) in said sample;

c) comparing the level of said biomarker to a reference level; and

d) determining whether the level of said biomarker is indicative of a risk of developing adverse cardiac remodeling,

wherein an increased level of said biomarker in said sample compared to the reference level is indicative of a risk of developing adverse cardiac remodeling.

Said subject may be at risk of developing heart failure and/or remote myocardial fibrosis.

The sample may be a biological sample such as a blood sample.

The reference level may be the level of said biomarker in a healthy subject.

The subject may have suffered from a myocardial infarction.

In a further aspect, the present invention pertains to a method for monitoring progression of adverse cardiac remodeling in a subject, said method comprising the steps of:

a) providing a first sample of said subject at a first time point, and at least a second sample of said subject at at least a second time point;

b) determining the level of at least one biomarker selected from P-selectin glycoprotein ligand-1 (PSGL-1) and integrin-alpha4 (CD49d) in said first sample and said at least a second sample;

c) comparing the level of said biomarker in said first sample to said at least a second sample; and

d) determining progression of adverse cardiac remodeling in said subject based upon the level of said biomarker between said first sample and said at least a second sample.

An identical level of said biomarker in said first sample and said at least a second sample may be indicative of arrest of adverse cardiac remodeling.

Said at least a second sample may be taken at a later time point than said first sample, and an increased level of said biomarker in said at least a second sample compared to the level of said biomarker in said first sample may be indicative of progression of adverse cardiac remodeling.

Said at least a second sample may be taken at a later time point than said first sample, and a decreased level of said biomarker in said at least a second sample compared to the level of said biomarker in said first sample may be indicative of decline in adverse cardiac remodeling.

In another aspect, the invention is concerned with a method for determining the response to therapy addressing adverse cardiac remodeling in a subject, said method comprising the steps of:

a) providing a first sample of said subject at a first time point, and a second at least a sample of said subject at at least a second time point;

b) determining the level of at least one biomarker selected from P-selectin glycoprotein ligand-1 (PSGL-1) and integrin-alpha4 (CD49d) in said first sample and said at least a second sample;

c) comparing the level of said biomarker in said first sample to said at least a second sample; and

d) determining the response to therapy addressing adverse cardiac remodeling in said subject based upon the level of said biomarker between said first sample and said at least a second sample.

An identical level of said biomarker in said first sample and said at least a second sample is indicative of a moderate response to therapy occurring with arrest of progression in adverse cardiac remodeling.

Said at least a second sample may be taken at a later time point than said first sample, and an increased level of said biomarker in said at least a second sample compared to the level of said biomarker in said first sample may be indicative of negative or low response to therapy.

Said at least a second sample may be taken at a later time point than said first sample, and a decreased level of said biomarker in said at least a second sample compared to the level of said biomarker in said first sample may be indicative of positive response to therapy.

Said therapy addressing adverse cardiac remodeling may be anti-fibronectin-EDA therapy.

Said anti-fibronectin-EDA therapy may be antibody therapy, wherein the antibody may be directed to the EDA domain, in whole or in part, of fibronectin-EDA.

Said antibody may be a monoclonal antibody, for example, a human, chimeric or humanized monoclonal antibody.

Said first sample and said at least a second sample may be biological samples such as blood samples.

Said adverse cardiac remodeling may result from myocardial infarction.

Said adverse cardiac remodeling may be indicative of the onset of heart failure and/or remote myocardial fibrosis.

In yet another aspect, the present invention is related to a kit comprising means for detecting PSGL-1 and CD49d in a sample obtained from a subject, preferably for use in identifying a subject at risk of developing adverse cardiac remodeling, diagnosing a subject suffering from adverse cardiac remodeling, monitoring progression of adverse cardiac remodeling, and/or determining the response to therapy addressing adverse cardiac remodeling in a sample obtained from a subject.

In an embodiment, said means are antibodies, preferably monoclonal antibodies.

### DEFINITIONS

As used herein, the term “adverse cardiac remodeling”, “adverse remodeling”, and “adverse ventricular remodeling” are used interchangeably. Remodeling is defined as alteration in the structure (dimensions, mass, shape) of the heart (called cardiac or ventricular remodeling) in response to hemodynamic load and/or cardiac injury in association with neurohormonal and inflammatory response activation. Remodeling may be described as physiologic or pathologic; alternatively, remodeling may be classified as adaptive or adverse (in the art also referred to as “maladaptive”). The adverse remodeling process frequently includes increases in myocardial mass. The heart can respond to environmental stimuli by growth (increased myocardial mass) or shrinkage (atrophy) with a dynamic range of at least 100 percent. Pathologic remodeling may occur with pressure overload (e.g. aortic stenosis, hypertension), volume overload (e.g. valvular regurgitation), or following cardiac injury (e.g. myocardial infarction, chronic ischemia, myocarditis), or in patients with genetic predispositions (idiopathic dilated cardiomyopathy; e.g. mutations in the MYH7, TNNT2 or SCN5A genes). In each of these settings, remodeling may transition from an apparently compensatory process to a maladaptive or adverse one. /pct

As used herein, the term “heart failure” refers to any condition characterized by decreased cardiac output and/or abnormal filling pressures in the ventricles. In these situations, the heart is unable to pump blood at an adequate rate or in adequate volume and/or in adequate force (i.e. systolic heart failure) or exhibits increased ventricular stiffness (i.e. diastolic heart failure), respectively. In heart failure, blood perfusion of organs is hampered thereby deteriorating organ function (e.g. kidney or liver failure). In addition, blood can back up into the lungs, causing the lungs to become congested with fluid. Typical symptoms of heart failure include shortness of breath (dyspnea), fatigue, weakness, difficulty breathing when lying flat, and swelling of the legs, ankles or abdomen (edema).

The term “antibody” used herein refers to any immunoglobulin or fragment thereof, and encompasses any polypeptide comprising an antigen-binding site with at least one complementarity determining region (CDR). The term includes, but is not limited to, polyclonal, monoclonal, monospecific, polyspecific, non-specific, humanized, chimeric, human, single-chain, single-domain (such as llama antibodies), synthetic, recombinant, hybrid, mutated, grafted and in vitro generated antibodies. The term “antibody” also includes antibody fragments such Fab, F(ab′)₂, Fv, scFv, Fd, dAb, and other antibody fragments or other constructs comprising CDRs that retain antigen-binding function. Typically, such fragments would comprise an antigen-binding domain. The details of the preparation of such antibodies and their suitability for use as binding members, particularly a specific binding member, are well known to those skilled in the art. The term “neutralizing” refers to the ability of an antibody to inhibit (i.e., eliminate or reduce) at least one activity of another compound or molecule. The antibody or fragment thereof may be any of the known antibody isotypes and their conformations , for example, IgA, such as IgA1 or IgA2, IgD, IgE, IgG, such as IgG1, IgG2a, IgG2b, IgG3, IgG4, or IgM class, or may constitute mixtures thereof in any combination, such as a mixture of antibodies from the IgG1 and IgG2a class.

### FIGURES

FIG. 1 shows CD49d (left-hand graph) and CD162 (right-hand graph) expression on circulating monocytes. The expression is significantly altered and different between fibronectin-EDA^{-/-} and WT mice after myocardial infarction. The alterations on monocytes precede the ventricular changes after infarction (28 days).

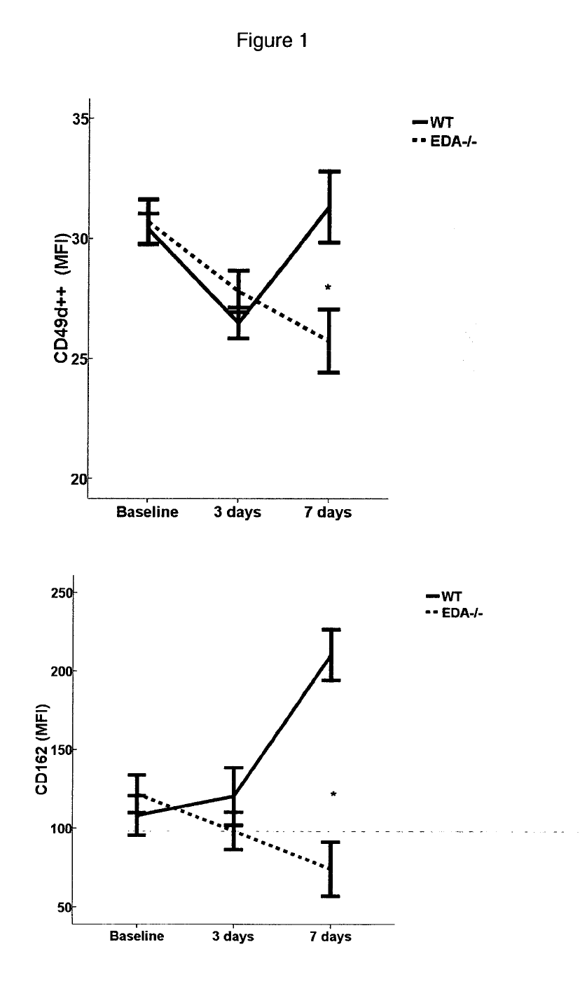

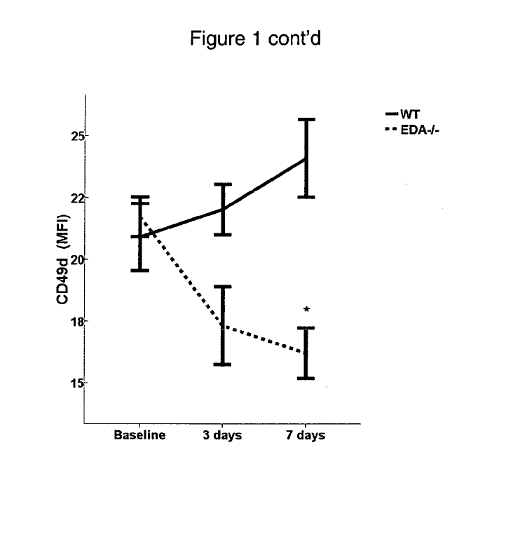

### DETAILED DESCRIPTION OF THE INVENTION

The present inventors have now identified two biomarkers for adverse cardiac remodeling. It was found that expression of P-selectin glycoprotein ligand-1 (PSGL-1; also referred to as “CD162” or “SELPLG”) and integrin-alpha4 (CD49d) on circulating white blood cells such as monocytes were significantly altered and different between fibronectin-EDA^{-/-} and WT mice, with the fibronectin-EDA^{-/-} mice being protected against adverse cardiac remodeling, after myocardial infarction. PSGL-1 and CD49d are associated with the inflammatory response mediated by fibronectin-EDA that is observed in the first few weeks after infarction. The local inflammatory reaction leading to the type of cardiac remodeling (adaptive vs. adverse) may be monitored on blood cells. If a subject suffering from myocardial infarction shows relatively high levels of PSGL-1 and/or CD49d, one can assume that the subject has entered the process of adverse cardiac remodeling and is at high risk of developing heart failure and/or other conditions associated with adverse cardiac remodeling such as remote cardiac fibrosis. Such subjects should be started on heart failure treatment before symptom onset with currently available or new adjunctive therapies.

The present invention relates to use of at least one biomarker selected from P-selectin glycoprotein ligand-1 (PSGL-1) and integrin-alpha4 (CD49d) for identifying a subject at risk of developing adverse cardiac remodeling, diagnosing a subject suffering from adverse cardiac remodeling, monitoring progression of adverse cardiac remodeling, and/or determining the response to therapy addressing adverse cardiac remodeling in a sample obtained from a subject.

Thus, the biomarkers may be used for identifying a subject at risk of developing adverse cardiac remodeling, and subsequent heart failure and/or remote myocardial fibrosis. The risk may be increased for such subject, in particular compared to healthy subjects. Healthy subjects as referred to herein do not suffer from any cardiac defects, and have not suffered cardiac injury. Preferably, a healthy subject does not suffer from any conditions or diseases.

Alternatively or additionally, the biomarkers may be used for diagnosing a subject suffering from adverse cardiac remodeling. As adverse cardiac remodeling is indicative of the onset of heart failure and/or remote myocardial fibrosis, a diagnosis of adverse cardiac remodeling would identify subjects at risk of developing diseases or conditions associated with such adverse cardiac remodeling, such as heart failure and/or remote myocardial fibrosis. Such subjects are most likely to have an increased risk of developing diseases or conditions associated with such adverse cardiac remodeling, such as heart failure and/or remote myocardial fibrosis. Diagnosis, as used herein, includes not only the initial identification of the occurrence of adverse cardiac remodeling but also confirmatory testing, or screening in subjects who have previously been identified as suffering from adverse cardiac remodeling or likely to suffer from adverse cardiac remodeling. A subject may have a greater likelihood of suffering from adverse cardiac remodeling or likely to suffer from adverse cardiac remodeling when he has a level of one or both of PSGL-1 and/or CD49d that is greater than the level in a reference. The level in the reference may be the mean biomarker(s) level in a population not suffering from adverse cardiac remodeling and/or not likely to suffer from adverse cardiac remodeling.

Alternatively or additionally, the biomarker(s) may be used for monitoring progression of adverse cardiac remodeling after onset by determining the level of one or both of the biomarkers PSGL-1 or CD49d at different time points after onset of cardiac injury or during the process of adverse cardiac remodeling in conditions such as volume overload, pressure overload and in patients with genetic predispositions. Adverse cardiac remodeling may have progressed if the level of said biomarker has increased in time. Alternatively, adverse cardiac remodeling may have been reversed if the level of said biomarker has decreased in time.

Alternatively or additionally, the biomarker(s) may be used for determining response to therapy addressing adverse cardiac remodeling. The therapy is preferably aimed at ameliorating or (partially) reversing the adverse cardiac remodeling process. The response may thus be therapeutic response to a drug (or, optionally, a combination of drugs) administered with the purpose of ameliorating or (partially) reversing the adverse cardiac remodeling process, e.g., for preventing onset of heart failure and/or remote myocardial fibrosis. The response to therapy may be determined by measuring the level of one or both of the biomarkers PSGL-1 or CD49d at different time points after start of therapy. One of the time points may be before the start of therapy. A positive response to therapy substantially means that adverse cardiac remodeling may have been reversed. A low or negative response to therapy substantially means that adverse cardiac remodeling progresses or continues. A moderate response to therapy substantially means that adverse cardiac remodeling is halted in its progression. The therapy addressing adverse cardiac remodeling may be therapy addressing the onset or occurrence of any disease or condition associated with adverse cardiac remodeling such as heart failure and/or remote myocardial fibrosis.

Said use is carried out ex vivo, i.e., on an ex vivo sample. Said sample may be any sample obtained from a subject, but is preferably a biological sample. As used herein, a “biological sample” refers to a biological tissue or biological fluid from a subject. A variety of samples can be useful in practicing the invention including, for example, blood, serum, plasma, urine, salivary fluid, ascite fluid, and the like. The sample preferably comprises white blood cells such as monocytes. Non-limiting examples of such sample is a whole blood sample. The term “biomarker(s)” as used herein refers to either one of PSGL-1 or CD49d alone, or both of PSGL-1 and CD49d.

The use of the invention encompasses ex vivo analyzing a sample for the level of one or both of the biomarkers of the invention, PSGL-1 and/or CD49d. The level of such biomarker or biomarkers may then be compared to a reference level. A reference level may be a level of said biomarkers or biomarkers in said subject prior to cardiac injury, e.g. myocardial infarction. Alternatively said reference level may be the level of said biomarker or biomarkers in a healthy subject or may be a default reference level determined by taking the average level of said biomarker(s) of a plurality of healthy subjects. It is not necessary to determine the level of said biomarker(s) each time a sample is measured; once the reference level of said biomarker(s) is reliably determined, the reference level may be stored, e.g., in a computer, and used for the comparative purposes herein set forth. Alternatively, the reference level may be known to the skilled physician and compared mentally. It is to be noted that the level of each biomarker is measured independently and compared to the reference level of the same biomarker. Moreover, PSGL-1 may be used as the single biomarker in the application of the present invention, CD49d may be used as the single biomarkers in the application of the present invention, or both PSGL-1 and CD49d may be used as biomarkers in the application of the present invention.

In the context of the invention, a subject may be an animal or a human being. In principle, any subject could be diagnosed using the method of the invention. The diagnosis method may be applied as often as necessary in a subject. Preferably, the subject is a human being. In a suitable embodiment, the subject is a subject having suffered cardiac injury such as myocardial infarction.

The adverse cardiac remodeling may be indicative of the onset and/or occurrence of heart failure and/or remote myocardial fibrosis.

In an aspect, the present invention is related to a method for identifying a subject at risk of developing adverse cardiac remodeling and/or diagnosing a subject suffering from adverse cardiac remodeling, said method comprising the steps of:

a) providing a biological sample of said subject;

b) determining the level of at least one biomarker selected from P-selectin glycoprotein ligand-1 (PSGL-1) and integrin-alpha4 (CD49d) in said sample;

c) comparing the level of said biomarker to a reference level; and

d) determining whether the level of said biomarker is indicative of a risk of developing adverse cardiac remodeling.

An increased level of said biomarker in said sample compared to the reference level may be indicative of a risk of developing adverse cardiac remodeling, such as an increased risk of developing adverse cardiac remodeling. A decreased, similar or identical level of said biomarker in said sample compared to the reference level may be indicative of no or a low risk of developing adverse cardiac remodeling. The reference level may be as described above.

The methods of the invention comprise the detection of one of PSGL-1 or CD49d, or both of PSGL-1 or CD49d.

The difference in the level of said biomarkers in said sample compared to a reference level may be determined as the fold increase/decrease, or alternatively as the relative increase or decrease of biomarker in the sample of said subject as compared to biomarker in a reference level.

In the methods of the invention, the level of said one or more proteins may be determined by methods well known in the art, e.g., using an enzyme immunoassay. Both PSGL-1 and CD49d are transmembrane proteins with a large extracellular domain. A number of monoclonal or polyclonal antibodies can be generated that specifically recognize PSGL-1 or CD49d. Alternatively, many commercially available antibodies against PSGL-1 can be purchased from, for example, Santa Cruz Biotechnology Inc. Antibodies against CD49d may, for example, be purchased from Miltenyi Biotec. Utilizing current antibody detection techniques that can quantitate the binding of monoclonal antibodies, made specifically to epitopes on the external domain of the transmembrane proteins, one can determine the level or amount of said PSGL-1 or CD49d in a sample obtained from a subject.

The term “enzyme immunoassay” (“EIA”), also called enzyme-linked immunosorbent assay (ELISA), is a biochemical technique that is well known in the art. It is used mainly in immunology to detect the presence of an antibody or an antigen in a sample. In ELISA, an unknown amount of antigen is affixed to a surface, and then a specific antibody is washed over the surface so that it can bind to the antigen. This antibody is linked to an enzyme, and in the final step a substance is added that the enzyme can convert to some detectable signal. Thus in the case of fluorescence ELISA, when light of the appropriate wavelength is shone upon the sample, any antigen/antibody complexes will fluorescence so that the amount of antigen in the sample can be inferred through the magnitude of the fluorescence signal.

Performing an ELISA involves at least one antibody or other binding partners with high affinity for the biomarker with specificity for a particular antigen. The sample with an unknown amount of antigen may be immobilized on a solid support (for example, a polystyrene microtiter plate) either non-specifically (via adsorption to the surface) or specifically (via capture by another antibody specific to the same antigen, in a “sandwich” ELISA). After the antigen is immobilized the detection antibody is added, forming a complex with the antigen. The detection antibody can be covalently linked to an enzyme, or can itself be detected by a secondary antibody which is linked to an enzyme through bioconjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. The substrates may utilize chromogenic substrates, though fluorogenic substrates and chemoluminescent substrates are used more commonly as they enable higher sensitivity. For some of the biomarkers of the invention, monoclonal antibodies may be commercially available from various suppliers all of which may be successfully applied, taking into account the manufacturers recommendations for use.

Alternatively, the amount of biomarker or biomarkers may be determined using any other routine techniques known to the skilled person, including, but not limited to, capillary action, precipitation, turbidimetric, diffusion, agglutination, potentiometric, amperometric, piezoelectric and evanescent-wave immunosensors, or any combination of the methods recited herein.

The skilled person will understand that instead of detecting the complete biomarker protein, one may also detect peptide fragments of said biomarker proteins, for example which are derived from the biomarker proteins by fragmentation thereof. The term peptide fragment as used herein refers to peptides having between 5 and 50 amino acids, for example 5, 10, 15, 20, 25, 30, 35, 40, 45 or 50 amino acids. These peptide fragments preferably provide a unique amino acid sequence of the protein, and are associated with the cardiovascular events as disclosed herein.

The proteins and/or peptide fragment may optionally be detected as chemically modified proteins and/or peptides, such chemical modification may for instance be selected from the group consisting of glycosylation, oxidation, (permanent) phosphorylation, reduction, myristylation, sulfation, acylation, acetylation, ADP-ribosylation, amidation, hydroxylation, iodination, and methylation. A large number of possible protein modifications is described in the RESID database at http://www.ebi.ac.uk/RESID (release 4 Jan. 2010) (Garavelli, J. S. (2004) The RESID Database of Protein Modifications as a resource and annotation tool; Proteomics 4: 1527-1533) and in Farriol-Mathis, N., Garavelli, J. S., Boeckmann, B., Duvaud, S., Gasteiger, E., Gateau, A., Veuthey, A., Bairoch, A. (2004) Annotation of post-translational modifications in the Swiss-Prot knowledge base. Proteomics 4(6): 1537-5. The skilled artisan is well aware of these modifications.

Also, the present invention relates to a method for monitoring progression of adverse cardiac remodeling in a subject, said method comprising the steps of:

a) providing a first sample of said subject at a first time point, and at least a second sample of said subject at a second time point;

b) determining the level of at least one biomarker selected from P-selectin glycoprotein ligand-1 (PSGL-1) and integrin-alpha4 (CD49d) in said first sample and said at least a second sample;

c) comparing the level of said biomarker in said first sample to said at least a second sample; and

d) determining progression of adverse cardiac remodeling in said subject based upon the level of said biomarker between said first sample and said at least a second sample.

The method comprising the steps of providing a first sample from said subject at a first time point, at least a second sample from said subject at a second time point, and determining the level of the biomarker(s) in both samples. By comparing these, progression of adverse cardiac remodeling in said subject can be determined. By measuring the level of biomarker(s) in a subject sample over time, a clinician will be able to determine whether the adverse cardiac remodeling has, for example, regressed, and whether the subject has been effectively treated. A clinician can therefore utilize this method for tailoring treatment appropriately. A subject whose adverse cardiac remodeling has regressed with an anti-adverse cardiac remodeling treatment will have a lower level of biomarker(s) than he did before treatment. Similarly, a subject whose adverse cardiac remodeling has remained stable during treatment will have similar levels of biomarker(s) as he did before treatment, and a subject whose adverse cardiac remodeling has worsened will have increased biomarker(s) levels at a later time point.

The term “at least a second sample” as used herein means that in addition to a second sample taken at a second time point, a third, fourth, fifth, sixth, seventh, eighth, ninth, tenth, or further sample may be taken at a third, fourth, fifth, sixth, seventh, eighth, ninth, tenth, or further time point to monitor progression of adverse cardiac remodeling in said subject. Assuming that a second time point is later than a first time point, a third time point is later than a second time point, and so on, a trend in progression of adverse cardiac remodeling may be demonstrated.

Additionally, the present invention pertains to a method for determining the response to therapy addressing adverse cardiac remodeling in a subject, said method comprising the steps of:

a) providing a first sample of said subject at a first time point, and at least a second sample of said subject at at least a second time point;

b) determining the level of at least one biomarker selected from P-selectin glycoprotein ligand-1 (PSGL-1) and integrin-alpha4 (CD49d) in said first sample and said at least a second sample;

c) comparing the level of said biomarker in said first sample to said at least a second sample; and

d) determining the response to therapy addressing adverse cardiac remodeling in said subject based upon the level of said biomarker between said first sample and said at least a second sample.

The term “therapy addressing adverse cardiac remodeling” as used herein refers to any treatment of any kind expected to ameliorate or reverse adverse cardiac remodeling in a subject.

The term “at least a second sample” as used herein means that in addition to a second sample taken at a second time point, a third, fourth, fifth, sixth, seventh, eighth, ninth, tenth, or further sample may be taken at a third, fourth, fifth, sixth, seventh, eighth, ninth, tenth, or further time point to determine the response to therapy addressing adverse cardiac remodeling in said subject. Assuming that a second time point is later than a first time point, a third time point is later than a second time point, and so on, a trend in the response to therapy addressing adverse cardiac remodeling in said subject.

The subject may be a positive responder, poor responder, or non-responder. For use herein, a positive responder is a subject who positively responds to treatment, i.e., a subject who experiences success in amelioration of adverse cardiac remodeling. A positive responder is one in which any toxic or detrimental side effects of pharmaceuticals are outweighed in clinical terms by therapeutically beneficial effects. A non-responder is a subject who does not respond to the treatment or does not respond to a satisfactory level. A poor responder is a subject who respond to treatment but not at the level of the positive responder.

A similar or identical level of said biomarker in said first sample and said at least a second sample may be indicative of a moderate response to therapy occurring with arrest of progression in adverse cardiac remodeling (poor responder). If said at least a second sample is taken at a later time point than said first sample, an increased level of said biomarker in said at least a second sample compared to the level of said biomarker in said first sample may be indicative of negative or low response to therapy (non-responder). If said at least a second sample is taken at a later time point than said first sample, a decreased level of said biomarker in said at least a second sample compared to the level of said biomarker in said first sample may be indicative of positive response to therapy (positive responder).

In certain embodiments, the therapy addressing adverse cardiac remodeling will include administration of an anti-adverse cardiac remodeling drug. A clinician or other suitable profession can use the information regarding a subject's likely response level to certain anti-adverse cardiac remodeling drug to determine an appropriate treatment regimen for the subject. In certain embodiments, increasing dosages can be provided for those subjects that are indicated to be poor responders or non-responders. In certain embodiments, subjects that are indicated to be poor responders or non-responders will receive a different class of drugs or therapy.

In an embodiment, the therapy addressing adverse cardiac remodeling may be anti-fibronectin-EDA therapy. Said anti-fibronectin-EDA therapy may be antibody therapy, wherein the antibody is directed and binds specifically to the EDA domain of fibronectin-EDA. Said antibody may be a monoclonal antibody, for example, a human, chimeric or humanized monoclonal antibody.

Said first and said at least a second sample may be blood samples. Said adverse cardiac remodeling may result from myocardial infarction. Said adverse cardiac remodeling may be indicative of the onset of and/or the occurrence of heart failure and/or remote myocardial fibrosis.

The invention also relates to a kit comprising means for detecting PSGL-1 and CD49d in a sample obtained from a subject. Said kit is preferably for use in identifying a subject at risk of developing adverse cardiac remodeling, diagnosing a subject suffering from adverse cardiac remodeling, monitoring progression of adverse cardiac remodeling, and/or determining the response to therapy addressing adverse cardiac remodeling in a sample obtained from a subject. Said means for detecting PSGL-1 and CD49d are preferably antibodies, preferably monoclonal antibodies. Said kit preferably comprises antibodies specifically binding to PSGL-1 and antibodies specifically binding to CD49d. The kit preferably comprises said antibodies in separate containers.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition of the invention may comprise additional component(s) than the ones specifically identified, said additional component(s) not altering the unique characteristics of the invention.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The terms “increased level” and “decreased level” as used throughout this document refers to a significantly increased level or significantly decreased level. Generally, a level in a test sample is increased or decreased when it is at least 5%, such as 10%, 15%, 20%, 25%, 30%, 35%, 40%, 45%, 50% higher or lower, respectively, than the corresponding level in a control sample or reference sample.

All patent and literature references cited in the present specification are hereby incorporated by reference in their entirety.

It will be clear that the above description and figures is included to illustrate some embodiments of the invention, and not to limit the scope of protection. Starting from this disclosure, many more embodiments will be evident to a skilled person which are within the scope of protection and the essence of this invention and which are obvious combinations of prior art techniques and the disclosure of this patent.

### EXAMPLE 1

### Methods

### Mice

Fibronectin-EDA knock out mice were generated as described previously by Tan et al. (Blood. 2004;104:11-18). EDA-/- mice were backcrossed into a Balb/C background for 8 generations.

### Flow Cytometry

TLR2, TLR4, CD49d and CD162 expression was assessed on circulating monocytes of EDTA anticoagulated blood by flow cytometry. Whole blood was stained for TLR2 (FITC, eBioscience, San Diego, Calif.), TLR4 (PE, eBioscience, San Diego, Calif.), CD49d (Alexa Fluor 488, Serotec, Oxford, UK), CD162 ((PE, BD Biosciences, Breda, the Netherlands) and F4/80 for monocytes (Alexa Fluor 647, Serotec, Oxford, UK).

Myocardial Infarction in vivo

Mice were anesthetized with a mixture of Fentanyl (Jansen-Cilag) 0.05 mg/kg, Dormicum (Roche) 5 mg/kg and medetomidine 0.5 mg/kg through an intraperitoneal injection. Core body temperature was maintained around 37° C. during surgery by continuous monitoring with a rectal thermometer and automatic heating blanket. Mice were intubated and ventilated (Harvard Apparatus Inc.) with 100% oxygen. The left coronary artery (LCA) was permanently ligated using an 8-0 vicryl suture. lschemia was confirmed by bleaching of the myocardium and ventricular tachyarrhythmia. In sham operated animals the suture was placed beneath the LCA without ligating. The chest wall was closed and the animals received subcutaneously Antisedan (Pfizer) 2.5 mg/kg, Anexate (Roche) 0.5 mg/kg and Temgesic (Schering-Plough) 0.1 mg/kg.

### Infarct Size

Infarct size (IS) as a percentage of the left ventricle (LV) was determined using Evans' blue dye injection and TTC staining, 2 days after infarction (n=6/group). By assessing infarct size in the acute phase (at 2 days), one can determine whether differences are present between WT and EDA-/- mice in myocardial perfusion. Hence, 4% Evans blue dye was injected via the thoracic aorta in a retrograde fashion. By doing so, one can demarcate the area-at-risk (AAR), the extent of myocardial tissue that underwent ischemia (i.e. endangered myocardium). Hearts were rapidly explanted, rinsed in 0.9% saline and put in −20° C. freezer for 1 hour. Hereafter, hearts were mechanically sliced into four 1-mm cross sections. Heart sections were incubated in 1% triphenyltetrazolium-chloride (Sigma-Aldrich) at 37° C. for 15 minutes before placing them in formaldehyde for another 15 minutes. Viable tissue stains red

and infarcted tissue appears white. Heart sections were digitally photographed (Canon EOS 400D) under a microscope (Carl Zeiss®). IS, AAR and total LV area were measured using

ImageJ software (version 1.34). Infarct size was corrected for the weight of the corresponding heart slice. After 28 days, IS/LV was determined using hematoxylin-eosin stained cross sections.

### Results

Lack of EDA Promotes Survival and Prevents Heart Function Deterioration as well as Maladaptive Remodeling After Myocardial Infarction

EDA synthesis is stimulated after infarction in both infarct and remote myocardium, reaching a peak at 7 and 3 days, respectively. Baseline MRI assessment of cardiac function and dimensions revealed no differences between EDA-/- and WT mice. Microscopic analyses did not show any alterations in cellularity and matrix composition in EDA-/- mice. The extent of endangered myocardium (AAR/LV) determined at 2 days post-MI was similar between the groups. Infarct size (IS) as percentage of LV was also similar between groups (IS/LV 38.2±1.2%, p=0.985. Kaplan-Meier survival analysis showed a significant survival benefit in EDA-/- mice over WTs. Most deaths occurred after day 6 and were not caused by cardiac ruptures (only 2 ruptures in the WT and 1 rupture in EDA-/- mice were observed during 28 days follow-up). In line with increased mortality, WT mice had greater LV dimensions and reduced systolic performance compared to EDA-/- mice. These significant differences were already present 7 days after infarction, and continued to deteriorate till 28 days post-MI. EDA-/- mice were relatively protected against remodeling and exhibited better systolic function after MI. The protective effect seen in EDA-/- was not attributable to changes in the extent of viable tissue, because infarct size did not differ between WT and EDA-/- mice 28 days post-MI (33.7±2.3% vs. 34.3±3.5%, respectively; p=0.818). However, wall thickness of the infarct area did not decline in EDA-/- mice as much as in WTs. At day 28 post-infarction, the entire infarct area was replaced by a dense collagen network in both groups. At day 7, however, there was reduced granulation of the infarct as shown by delayed degradation of acellular matrix in EDA-/- mice.

### EDA-/- Mice Exhibit Less Post-Infarct Fibrosis

Collagen deposition occurs in the infarct area upon degradation of the matrix and in the remote myocardium upon changes in wall stress. Myofibroblasts are the primary source of de novo collagen synthesis. In our study, collagen deposition in both the infarct and remote area was similar between the groups at day 7 post-MI. After 28 days, collagen content was again similar in the infarct area, suggesting that scar formation is not negatively affected in EDA-/- mice. However, the remote myocardium now contained less collagen fibers in EDA-/- mice compared to WT animals. These findings were supported at the mRNA level. Both procollagen-1 and -3 are reduced in the remote myocardium of EDA-/- mice. Within the infarct, collagen synthesis in EDA-/- mice was again comparable to WT animals. There was no difference in lysyl-oxidase and TIMP-2 production between the groups, suggesting no differences in collagen cross-linking and protease inhibition, respectively. The reduced fibrosis in the remote myocardium at 28 days post-MI is preceded by a significantly decreased myofibroblast transdifferentiation in EDA-/- mice, in both remote and infarct areas 7 days after infarction. Periostin is described as a maturation factor of cardiac fibroblasts. In our study, periostin-positive area was reduced as well in EDA-/- mice compared to WT animals. To study whether WT and EDA-/- myofibroblasts differed in their matrix synthesis activity and MMP expression profile, we cultured post-infarct myofibroblasts and stained for myofibroblast markers and pro-collagen-III. In vitro, there were no differences between the two genotypes. In addition, zymography was done using the supernatants of the cells and showed also no differences in MMP2 and -9 activity.

### Lack of EDA Results in Enhanced Inotropy and Lusitropy

Altered fibrotic processes in EDA-/- mice indicate that diastolic function could be affected as well. Contractility, as indicated by dP/dTmax, was much higher in EDA-/- mice after 28 days infarction. This confirmed our previous MRI findings, that EDA-/- mice exhibit enhanced systolic performance. Increase of LVEDP and tau is detrimental for heart function and is caused by increase of EDV and/or fibrosis; one of the hallmarks of heart failure. Compared to WT animals, diastolic performance was also significantly enhanced in EDA-/- mice after 28 days infarction. Both parameters were significantly lower in EDA-/- mice compared to WT animals, providing evidence that the improved survival in EDA-/- mice is a consequence of both systolic and diastolic functional improvements.

### EDA Regulates Post-MI Inflammation

Considered as a ligand for TLR2 and 4, lack of EDA should result in a decreased inflammatory status. Neutrophils are the first leukocyte subset migrating upon tissue injury and are known to be associated with the extent of damage. Neutrophil count in the infarct area was not different between the groups. Hereafter, macrophages clear cell debris (e.g. necrotic neutrophils and cardiomyocytes) and, more importantly, initiate the remodeling process after infarction. In our study, the number of macrophages was highly reduced in EDA-/- mice 7 days post-infarction. There were no cells detectable after 28 days infarction in both groups. In concordance with the reduced macrophage influx, levels of TNFα, RANTES, GM-CSF (responsible for recruitment, differentiation and maturation of macrophages) and IL-10 were highly reduced in EDA-/- mice, 7 days after infarction. In contrast, MCP-1 levels were increased in EDA-/- mice compared to WT animals at protein and mRNA level.

EDA mediates both Integrin-α4, Toll-Like Receptor and CD 162 Expression in Circulating Monocytes

EDA is a known ligand for integrin-α4β1 (VLA-4) and TLR2 and 4. Since parenchymal EDA mediates adverse remodeling, we hypothesized that EDA from the heart may serve as an endogenous activator of circulating cells after infarction. EDA-/- mice showed a significant reduction in peripheral monocytes 3 days after infarction, whereas after 7 days the numbers were similar between the groups. TLR2 expression on monocytes was significantly altered in the absence of EDA, while TLR4 did not show any difference in expression levels after MI between the groups. Integrin-α4 (CD49d) expression and CD162 were also significantly reduced on monocytes of EDA-/- mice after infarction. In addition, there was a subgroup of monocytes that showed a significant higher expression level of CD49d. EDA-/- mice showed again a reduced CD49d expression in this subgroup, 7 days post-infarction.

1.-3. (canceled)

4. A method for identifying a subject at risk of developing adverse cardiac remodeling and/or diagnosing a subject suffering from adverse cardiac remodeling, said method comprising the steps of:
a) providing a sample of said subject;
b) determining the level of at least one biomarker selected from P-selectin glycoprotein ligand-1 (PSGL-1) and integrin-alpha4 (CD49d) in said sample;
c) comparing the level of said biomarker to a reference level; and
d) determining whether the level of said biomarker is indicative of a risk of developing adverse cardiac remodeling,
wherein an increased level of said biomarker in said sample compared to the reference level is indicative of a risk of developing adverse cardiac remodeling.

5. The method according to claim 4, wherein said subject is further at risk of developing a myocardial infarction-related condition.

6. The method according to claim 4, wherein said subject is at risk of developing heart failure and/or remote myocardial fibrosis.

7. The method according to claim 4, wherein the sample is a blood sample or is derived from a blood sample.

8. The method according to claim 4, wherein the reference level is the level of said biomarker in a healthy subject.

9. The method according to claim 4, wherein the subject has suffered from myocardial infarction.

10. A method for monitoring progression of adverse cardiac remodeling in a subject, said method comprising the steps of:
a) providing a first sample of said subject at a first time point, and at least a second sample of said subject at at least a second time point;
b) determining the level of at least one biomarker selected from P-selectin glycoprotein ligand-1 (PSGL-1) and integrin-alpha4 (CD49d) in said first sample and said at least a second sample;
c) comparing the level of said biomarker in said first sample to said at least a second sample; and
d) determining progression of adverse cardiac remodeling in said subject based upon the level of said biomarker between said first sample and said at least a second sample.

11. The method according to claim 10, wherein an identical level of said biomarker in said first and at least a second sample is indicative of arrest of adverse cardiac remodeling.

12. The method according to claim 10, wherein said at least a second sample is taken at a later time point than said first sample, and wherein an increased level of said biomarker in said at least a second sample compared to the level of said biomarker in said first sample is indicative of progression of adverse cardiac remodeling.

13. The method according to claim 10, wherein said at least a second sample is taken at a later time point than said first sample, and wherein an decreased level of said biomarker in said at least a second sample compared to the level of said biomarker in said first sample is indicative of decline in adverse cardiac remodeling.

14. A method for determining the response to therapy addressing adverse cardiac remodeling in a subject, said method comprising the steps of:
a) providing a first sample of said subject at a first time point, and at least a second sample of said subject at at least a second time point;
b) determining the level of at least one biomarker selected from P-selectin glycoprotein ligand-1 (PSGL-1) and integrin-alpha4 (CD49d) in said first sample and said at least a second sample;
c) comparing the level of said biomarker in said first sample to said at least a second sample; and
d) determining the response to therapy addressing adverse cardiac remodeling in said subject based upon the level of said biomarker between said first sample and said at least a second sample.

15. The method according to claim 14, wherein an identical level of said biomarker in said first sample and said at least a second sample is indicative of a moderate response to therapy occurring with arrest of progression in adverse cardiac remodeling.

16. The method according to claim 14, wherein said at least a second sample is taken at a later time point than said first sample, and wherein an increased level of said biomarker in said at least a second sample compared to the level of said biomarker in said first sample is indicative of negative or low response to therapy.

17. The method according to claim 14, wherein said at least a second sample is taken at a later time point than said first sample, and wherein a decreased level of said biomarker in said at least a second sample compared to the level of said biomarker in said first sample is indicative of positive response to therapy.

18. The method according to claim 14, wherein said therapy addressing adverse cardiac remodeling is anti-fibronectin-EDA therapy.

19. The method according to claim 18, wherein said anti-fibronectin-EDA therapy is antibody therapy, wherein the antibody is directed to the EDA domain of fibronectin-EDA.

20. The method according to claim 19, wherein said antibody is a monoclonal antibody, for example, a human or humanized monoclonal antibody.

21. The method according to claim 10, wherein said first sample and said at least a second sample are blood samples or samples derived from blood.

22. The method according to claim 10, wherein said adverse cardiac remodeling results from myocardial infarction.

23. The method according to claim 10, wherein said adverse cardiac remodeling is indicative of the onset of and/or the occurrence of heart failure and/or remote myocardial fibrosis.

24. A kit comprising means for detecting PSGL-1 and CD49d in a sample obtained from a subject.

25. The kit according to claim 24 for use in identifying a subject at risk of developing adverse cardiac remodeling, diagnosing a subject suffering from adverse cardiac remodeling, monitoring progression of adverse cardiac remodeling, and/or determining the response to therapy addressing adverse cardiac remodeling in a sample obtained from a subject.

26. The kit according to claim 24, wherein said means are antibodies, preferably monoclonal antibodies.
